(19) United States
(12) Patent Application Publication
(10) Pub. No.: US 2026/0026929 A1
(43) Pub. Date: Jan. 29, 2026

(54) OSSICULAR PROSTHESIS SELECTABLE FROM A SINGLE ARRANGEMENT

(71) Applicant: Heinz Kurz GmbH, Dusslingen (DE)

(72) Inventors: Alexander Sander, Titisee-Neustadt (DE); Thomas Stiegele, Riederich (DE); Matthias Mertens, Kaltenkirchen (DE)

(73) Assignee: Heinz Kurz GmbH, Dusslingen (DE)

(21) Appl. No.: 19/257,460

(22) Filed: Jul. 1, 2025

(30) Foreign Application Priority Data
Jul. 25, 2024 (DE) 102024121204.7

(51) Int. Cl. A61F 2/18

(52) U.S. Cl. CPC A61F 2/18; A61F 2002/183; A61F 2220/0008; A61F 2220/0033; A61F 2230/0069; A61F 2230/0093; A61F 2250/0037

(57) ABSTRACT

An ossicular prosthesis having a first fastening element designed as a head plate for contact with the tympanic membrane, a second fastening element for mechanical connection to a member of the ossicular chain or the inner ear, and a shaft-shaped connecting element connecting the two fastening elements in a manner conducting sound. The first fastening element has a through-bore for receiving the connecting element and can be narrowed to provide fixation. The connecting element protrudes through the through-bore onto and carries a third fastening element designed for connection to a member of the ossicular chain or the inner ear. The connecting element can be easily separated above or below the first fastening element. This arrangement offers middle ear surgeons a simple and quick option to choose from a range of different prosthesis types immediately before inserting a selected prosthesis into the patient's middle ear.

### CROSS-REFERENCE TO RELATED APPLICATIONS

This application claims priority to German Patent Application No. DE 10 2024 121 204.7 filed on Jul. 25, 2024, the entire contents of which are hereby incorporated in full by this reference.

### FIELD OF THE INVENTION

The invention relates to an ossicular prosthesis which replaces or bridges at least one member or parts of a member of the ossicular chain, the ossicular prosthesis comprising a first fastening element designed as a flat head plate for mechanical engagement with the tympanic membrane, a second fastening element for mechanical connection to a member or parts of a member of the ossicular chain or to the inner ear, and a shaft-shaped connecting element which connects the two fastening elements to one another along a longitudinal axis in a manner conducting sound, and the plate-shaped first fastening element having a through-bore in its center for receiving the shaft-shaped connecting element, which through-bore is designed to be able to be narrowed for fixing the connecting element.

### BACKGROUND OF THE INVENTION

Such a device is known from DE 298 19 892 U1≈EP 0 998 884 B1≈U.S. Pat. No. 6,387,128 B1 (reference [1]).

Ossicular prostheses are used to transfer the sound from the tympanic membrane to the inner ear in the case of completely or partially missing or damaged ossicles of the human middle ear. In this case, the ossicular prosthesis has two ends, depending on the specific circumstances one end of the ossicular prosthesis resting, for example by means of a head plate, on the tympanic membrane or the handle of malleus, and the other end of the ossicular prosthesis being fastened, for example, to the stirrup bone of the human ossicular chain or being dipped directly into the inner ear. In many cases, the known ossicular prostheses allow sound conduction between the tympanic membrane and the inner ear only to a limited extent because they can only replace the natural anatomical formations of the ossicular chain in a very limited manner. (See, for example, DE 42 10 235 C1; EP 0 809 982 B1 (reference [2])).

After the prosthesis has been surgically placed in the middle ear and the tympanic membrane is closed again, the so-called healing phase begins. During this time, scars are formed, and they cause unpredictable forces which may result in the prosthesis moving from its local position. In the case of a rigid connection between the head plate and the shaft, increased pressure peaks can occur between the edge of the head plate and the tympanic membrane or the graft between the tympanic membrane and the head plate. These can be so high that penetration or extrusion through the tympanic membrane may result. For this reason, it is very helpful if the prosthesis has a certain post-operative mobility so that the head plate can automatically adapt to the position of the tympanic membrane post-operatively.

Moreover, since the anatomical conditions of the ear, such as the position, the shape and the size of the stirrup bone, the anvil, the hammer and the tympanic membrane vary, it is very advantageous if ossicular prostheses are not designed to be rigid but have a certain flexibility or variability. In order to achieve such flexibility/variability, various fastening and coupling devices for ossicles, which have resilient parts and/or joints, are known. Such an articulated connection between a fastening element, mountable on the stirrup foot plate, and the elongate shaft is described for example in EP 1 181 907 B1 (=reference [3]) and is offered by the applicant under the brand name “Ball Joint”.

A further complication, which occurs in various ways, arises due to underventilation of the middle ear space and associated inflammation, tumor formations, adhesive processes in the tympanic membrane region and stiffening of the latter. For example, in the case of a dysfunction of the Eustachian tube, a negative pressure can occur in the middle ear, which can cause a bulge or protrusion (known as retraction) of the tympanic membrane and consequently adhesion for example to the stirrup bone. In order to counteract this and to be able to follow postoperative movements of the tympanic membrane, in known ossicular prostheses, the head plates are designed to be tiltable relative to the connecting element which connects the head plate to the second fastening element and is designed in most cases as an elongate shaft. Such a head plate which is inherently rigid but tiltable relative to the connecting element is described, inter alia, in US 2004/0162614 A1 (=reference [4]).

While reference [3] has an ossicular prosthesis in which the head plate has a built-in ball joint connected to the connecting shaft of the two fastening elements, EP 1 833 424 B1 (=reference [5]) even discloses a connecting shaft designed as a ball chain. As a result, the final axial length of the prosthesis can be set, at least in a stepped manner, by the selection of a certain number of balls in the chain and cutting off the excess balls.

However, a disadvantage of these known ossicular prostheses is that, due to the rigid tilting of the head plate during local medial movements of the tympanic membrane, the opposite side of the head plate is simultaneously moved laterally outwards, whereby pressure peaks are generated on the tympanic membrane.

EP 1 972 307 B1 (=reference [6]) discloses an ossicular prosthesis with a head plate designed to be highly flexible in which, on the one hand, the advantages of the prosthesis according to the above-mentioned reference [5] and also, on the other hand, the advantages of the prostheses described in reference [4] are maintained, but the common disadvantages of a rigid tilting of the head plate are prevented. A problem with the ossicular prosthesis according to reference [6], however, is its insertability into the middle ear of the patient because, in particular, in the radial direction with respect to its longitudinal axis, the head plate projects significantly laterally and can only be surgically inserted into the middle ear through the tympanic membrane region by means of a large artificial opening. This large opening then of course also grows closed post-operatively with difficulty and also leaves correspondingly large scars.

The head plate comprises, in reference [6], flexible rib elements which run within the head plate plane and connect a radially outer annular region of the head plate to a central coupling region arranged radially in the center of the head plate. These rib elements, together with the radially outer annular region and the central coupling region, are a fixed component of the head plate disclosed in the reference [6]. They are geometrically designed in such a way that they follow such a medial movement—also locally—during local medial movements of the tympanic membrane, but do not transmit the movement to remote regions of the head plate.

However, the rib elements are not designed for any radial compression of the head plate within the head plate plane and therefore a radial reduction of the head plate diameter, possibly for the purpose of facilitating passage through the tympanic membrane and would be unsuitable for such a use. This is, in particular, because the radially outer annular region is rigid and therefore does not follow the attempt of such radial compression or would at most bend uncontrollably.

The ossicular prosthesis described in reference [6] is also entirely unsuitable for a change in length in the direction of the z-axis because, although the connecting element, which extends along the z-axis and rigidly connects the head plate to the second fastening element, is designed to transmit sound, it is designed as such to be rigid. Flexibility of the length of the connecting element in the z-direction is excluded and also not desired at all in the ossicular prosthesis according to reference [6].

EP 3 311 773 B1 (=reference [7]), in contrast, describes an ossicular prosthesis with a head plate that can be folded like an umbrella, and by means of which the prosthesis can be inserted into the middle ear in a virtually minimally invasive manner through a small opening in the tympanic membrane.

Another important issue in the implantation of ossicular prostheses is the setting of the correct axial length of the prosthesis that is optimally adapted to the individual circumstances and geometric conditions in the middle ear of the patient.

Reference [5], discussed above, already proposes a ball chain having separable end balls for this purpose. However, this unfortunately does not allow continuous, but merely a stepwise, length adjustment.

A length-variable ossicular prosthesis having a displacement mechanism, which is installed in the connecting element between the first and second fastening elements, for stepless length adjustment is described in DE 10 2007 041 539 B4 (=reference [8]).

Instead of such a displacement mechanism, which is relatively complex to produce, EP 2 238 946 B1 (=reference [9]) proposes a length-variable ossicular prosthesis in which an accordion-like structure is installed in the connecting element. The axial length of the ossicular prosthesis can then be shortened by an axial compression of this structure and increased by pulling it out.

EP 2 601 909 B1 (=reference [10]) in turn provides a displacement mechanism in the connecting element for the axial length adjustment of the ossicular prosthesis, which displacement mechanism comprises a receiving part and an insertion part which surrounds the receiving part with two legs in a clamp-like manner, the receiving part and the insertion part being displaceable relative to one another in the axial direction of the connecting element.

A laser-activatable length-variable ossicular prosthesis is disclosed in EP 3 130 315 B1 (reference [11]). It is proposed therein to construct the connecting element having stretchable and/or compressible partial strands, folded in a loop-like manner, which are made of a shape-memory material. Activation surfaces are connected to the loops of these partial strands in a heat-conducting manner, which surfaces can cause thermal activation, and therefore a deformation of the loops, by the action of heat. In this way, the axial length of the ossicular prosthesis can be changed and adjusted as desired.

EP 1 961 400 B1 (reference [12]) describes a partial ossicular prosthesis, at one end of which a loop is provided, by means of which a region of a member of the ossicular chain can be gripped with a non-positive connection, the loop being designed in such a way that, in the implanted state of the prosthesis, it rests firmly on this member, but only in subregions thereof, in order to prevent complete strangulation of the coupling point on the auditory ossicle. In addition, reference proposes to manufacture at least one of the fastening elements from a material with shape memory (memory effect) or superelastic properties, preferably from Nitinol.

DE 10 2022 119 451 B3 (reference [13]) presents an ossicular prosthesis with in-situ elongation, in which the prosthesis is constructed in two parts, namely a head plate on the one hand and the connecting element with a second fastening element on the other. The head plate has clamp-like arms by means of which it can be coupled to grooves that run azimuthally around the connecting element. This allows the length of the ossicular prosthesis to be adjusted even after implantation.

From DE 10 2009 006 047 B3 (reference [14]), a passive ossicular prosthesis with an applicator is known, which is characterized in that a longish applicator is provided for the transport of the ossicular prosthesis from a sterile package to the field of operation and placing into the middle ear or into the auditory canal, which applicator on the one hand is featuring a free end for handling sticking out of the ossicular prosthesis, on the other hand is featuring an engagement part, that is initially fastened at the ossicular prosthesis in a force-fit or form-fit manner or via a breakable or shearable material bridge, and after placing the ossicular prosthesis into the middle ear or the auditory canal can be separated from the ossicular prosthesis and can be removed from the middle ear or the auditory canal together with the applicator. This facilitates a simple and cost-efficient provisioning of a unit comprising both, an ossicular prosthesis and an individual transporting means being optimally adapted to the geometry of the ossicular prosthesis, which unit can be withdrawn from a sterile packing ready for use in a surgical operation. After implantation of the prosthesis, the applicator can be easily removed from the middle ear and be disposed, which enormously facilitates the practical handling of the ossicular prosthesis for the surgeon. The whole process is standardized and no other auxiliary means are necessary except for the operational microscope, in particular, no sterile pre-treatment of the equipment.

DE 10 2020 108 887 A1 (reference [15]) describes an ossicular prosthesis the length of which can be adjusted with a tympanic head plate designed as an elliptical ring having a locking element that can lock the head plate fixed at the connecting element at different axial positions of the shaft-shaped connecting element.

Finally, US 2020/0113675 A1 (reference [16]) shows a middle ear prosthesis having a tympanic head plate coated with bio-compatible silicone, where the coating is integrated in the head plate in such a way that it touches the tympanum at least partly in the implanted state of the prosthesis.

The parallel patent publications of reference [1] cited above disclose a generic ossicular prosthesis having all of the sets of features defined at the outset. In particular, they propose to provide strip-like rib elements of the generic ossicular prosthesis in the connecting element, which rib elements can be spread radially outwards away from the longitudinal axis and in the process shorten the axial length of the connecting element and therefore the axial length of the ossicular prosthesis. The rib elements are spread from the side, i.e., in the radial direction with respect to the connecting element.

### SUMMARY OF THE INVENTION

### Object of the Invention

In contrast, the object of the present invention is to improve a generic ossicular prosthesis of the type defined at the outset using the simplest possible technical means in such a way that it is easy and cost-effective to create a simple and quick option for middle ear surgeons to select from a series of different types of ossicular prostheses immediately before inserting the prosthesis specifically selected by the surgeon into the patient's middle ear. Such a choice is not provided or even hinted at in any of the references [1] to [13] discussed above.

### BRIEF DESCRIPTION OF THE INVENTION

According to the invention, this object is achieved in a surprisingly simple and effective manner in that the shaft-shaped connecting element protrudes through the through-bore onto the side of the first fastening element opposite the second fastening element, in that the connecting element carries a third fastening element at its free end opposite the second fastening element, which third fastening element is designed for mechanical connection to a member or parts of a member of the ossicular chain or to the inner ear, and in that the shaft-shaped connecting element is designed, at least in its two regions adjacent on both sides to the through-bore of the first fastening element, in such a way that it can be broken off above or below the plate-shaped first fastening element or can be separated by means of a separation tool.

This allows the advantages of the generic ossicular prosthesis described above, as described in reference [1], to be used in a relatively simple manner, middle ear surgeons now having the option, without further preparation, of selecting one of the two different configurations of the ossicular prosthesis offered with the arrangement according to the invention directly during the operation—in particular, after visual inspection of the specific individual situation in the patient's middle ear—shortly before inserting the prosthesis, in order to provide the patient with optimal care.

The surgeon can very easily separate the non-selected, i.e., discarded, part of the arrangement at the shaft piece which extends through the head plate (first fastening element), with the unwanted third fastening element attached to it above the head plate, for example by simply breaking it off or using a separation tool.

This leaves the ossicular prosthesis with the configuration selected by the surgeon of head plate, connecting element and the desired second fastening element attached thereto as a complete and immediately usable ossicular prosthesis, and is immediately available for implantation without further modifications.

If the arrangement according to the invention contains a third fastening element that is functionally different from the second fastening element, the surgeon can select the optimal second fastening element for the actual situation found in the patient's middle ear without any time delay and can implant it immediately after removing the third fastening element.

However, if the arrangement according to the invention contains two functionally similar fastening elements (in addition to the always-present head plate), but the shaft length between the head plate and the second fastening element is different from the shaft length between the head plate and the third fastening element, the surgeon can easily adjust the desired prosthesis length by making the appropriate selection.

In any case, the present invention provides the attending physician with significantly greater flexibility in middle ear surgery.

In addition, the need to simultaneously store many different middle ear prostheses is significantly reduced.

### Preferred Embodiments of the Invention

The arrangement according to the invention having a shaft-shaped connecting element and a flat head plate for mechanical attachment to the tympanic membrane as central components, as well as a second fastening element at one end of the shaft and a third fastening element at the opposite end of the shaft, basically does not require any further “ingredients.”

However, in order to facilitate the separation of the non-selected part of the arrangement by breaking it off, a particularly preferred class of embodiments of the ossicular prosthesis according to the invention is characterized in that the shaft-shaped connecting element has a predetermined breaking point in each of its two regions adjacent to the through-bore of the first fastening element on both sides thereof.

In a preferred implementation of this class of embodiments, the predetermined breaking point comprises an annular, azimuthally circumferential material recess around the shaft-shaped connecting element. Such a predetermined breaking point can be created by turning or milling the annular material recess and does not require any special selection of the bending direction for breaking off the unwanted part of the prosthesis. Rather, the bending can occur in any radial direction with respect to the shaft axis.

Alternatively, in a further implementation, a notch- or wedge-shaped, one-sided radial material recess is provided in the shaft-shaped connecting element as a predetermined breaking point above and below the shaft-shaped connecting element. Such a notch or wedge can be produced on the side of the shaft in a technically extremely simple and inexpensive manner but then specifies a preferred radial direction when bending.

A first group of embodiments of the arrangement according to the invention is characterized in that the first fastening element together with the second fastening element and with a first part of the shaft-shaped connecting element located therebetween forms a total prosthesis, while the first fastening element together with the third fastening element and with a second part of the shaft-shaped connecting element located therebetween forms a partial prosthesis.

An advantage of this configuration is that middle ear surgeons only have to make a final decision immediately before inserting the final middle ear prosthesis as to whether, in view of the actual situation in the patient's middle ear, a total prosthesis is required that bridges all parts of the auditory ossicles, or whether there is still sufficient substance in one or more auditory ossicles to insert a partial prosthesis.

In a second group of embodiments of the invention, the first fastening element together with the second fastening element and with a first part of the shaft-shaped connecting element located therebetween forms a partial prosthesis, while the first fastening element together with the third fastening element and with a second part of the shaft-shaped connecting element located therebetween also forms a partial prosthesis.

When this arrangement is used, it has already been decided that the ossicular prosthesis to be implanted will be a partial prosthesis. However, the treating surgeon can still select the type of second fastening element at short notice.

If it has already been established—for example through preliminary examinations of the patient—that the auditory ossicles are already so damaged that a partial prosthesis is no longer an option and a total prosthesis must be used, the treating surgeon can still select the type of the selected second fastening element in a third group of embodiments of the arrangement according to the invention: In this case, the first fastening element together with the second fastening element and with a first part of the shaft-shaped connecting element located therebetween forms a total prosthesis, and the first fastening element together with the third fastening element and with a second part of the shaft-shaped connecting element located therebetween also forms a total prosthesis.

Further implementations of the second and third group of embodiments described above, which are characterized in that the first part of the shaft-shaped connecting element between the first fastening element and the second fastening element has a different axial length than the second part of the shaft-shaped connecting element between the first and the third fastening element, are advantageous. In this way, once it has been determined whether a partial or total prosthesis is to be used, the treating surgeon can easily pre-select the approximate length.

These implementations can be further improved in their practical suitability by the first part of the shaft-shaped connecting element having an axial length between 1.75 mm and 3.5 mm, preferably 2 mm, and by the second part of the shaft-shaped connecting element having an axial length between 3 mm and 7 mm, preferably 4 mm.

In practice, embodiments of the ossicular prosthesis according to the invention have also proven successful in which the shaft-shaped connecting element has a radial thickness transverse to the longitudinal axis between 0.1 mm and 0.2 mm, preferably 0.18 mm.

By selecting the exact type of second (or third) fastening element required, the treating surgeon determines the optimal design of the ossicular prosthesis used for the given patient.

For example, such embodiments of the arrangement according to the invention are characterized in that the second fastening element and/or the third fastening element is designed as a slotted bell.

Further advantageous embodiments of the invention are characterized in that the second fastening element and/or the third fastening element is designed as a clip.

Embodiments of the arrangement according to the invention in which the second fastening element and/or the third fastening element is stem-shaped can also be advantageous.

Embodiments in which the second fastening element and/or the third fastening element is designed as a piston are also possible and may be preferred.

The special effects and advantages of these four variants mentioned above are explained in more detail in the detailed description below.

In summary, in further embodiments of the invention, the second fastening element can be designed as a plate, in particular, as a curved plate, as a sleeve, as a loop, as a closed bell, in particular, in the form of a hollow cylinder, as a singly or multiply slotted bell, or as a clip for mechanical connection to a further member of the ossicular chain. Alternative embodiments can provide for the ossicular prosthesis to be coupled directly to the inner ear, at its end supporting the second fastening element, by perforating the stirrup foot plate (=stapedectomy or stapedotomy) and/or by opening the human cochlea (=cochleostomy), in particular, by means of a piston.

In addition to the post-operative position shift, there is also a further problem after the implantation of ossicular prostheses, specifically that the middle ear of the human body represents a “half-open site.” Each implantation material which is introduced into the body in the context of a reconstruction of the middle ear and its structures, thereby experiences a particular stress due to the fact that a contaminated and infected environment prevails, which usually attacks the material. Since the aim of the implantation of an ossicular prosthesis must also always be to keep the implant in the middle ear of the patient for as long as possible without complications, a prolonged attack on the material can lead to damage to the prosthesis and/or to local infection. Both consequences are not tolerable. In order to permanently prevent damage to both the implantation material and the surrounding tissue, in a further particularly preferred embodiment of the invention, the surface of the ossicular prosthesis is completely or at least sectionally coated with a biologically active coating, in particular, a growth-inhibiting and/or a growth-promoting and/or an antibacterial coating. The head plate of the ossicular prosthesis according to the invention should in principle have a growth-promoting coating, while a second fastening element, which leads directly into the inner ear and is designed for example in the form of a piston, has a growth-inhibiting coating.

The use of biocompatible materials such as gold or titanium, which are often used in ossicular prostheses, reduces the risk of postoperative infections and rejection reactions.

Alternatively or additionally, in further embodiments, parts of the ossicular prosthesis according to the invention can be made from a ceramic material. However, embodiments of the invention are also possible in which the prosthesis or parts thereof are made of biocompatible plastics, in particular, silicone, polytetrafluoroethylene (PTFE) or fiber composite materials. These materials can also prevent post-operative rejection reactions in most cases.

The scope of the present invention also includes a system comprising an ossicular prosthesis constructed according to the invention of the type described above, and a separation tool for cutting through the shaft-shaped connecting element.

The separation tool can be designed as cutting pliers to form a defined tip at the separation point. With their help, the surgeon removes the protruding prosthetic shaft with the unselected third fastening element on the lateral side of the tympanic head plate. A spike or pin is created which, when the ossicular prosthesis is inserted, fixes the transplant material, such as cartilage or fascia, placed between the implant and the tympanic membrane during the operation.

The separation tool can serve as an implantation aid, especially for adjusting the length of the selected ossicular prosthesis. It can be designed as a minimally invasive, in particular, endoscopic, instrument, preferably tweezer-like or pincer-like.

### BRIEF DESCRIPTION OF THE DRAWINGS

The schematic drawing shows embodiments of the invention which are explained in greater detail in the following description.

In the drawings:

FIG. 1a is a schematic side view of a first embodiment of the arrangement according to the invention, comprising a shaft-shaped connecting element protruding through the first fastening element designed as a tympanic head plate, with a second fastening element designed as a slotted bell at one end and a third fastening element designed as a stem at the other end, the first part of the shaft between the bell and the head plate having the same axial length as the second part of the shaft between the stem and the head plate;

FIG. 1b is a three-dimensional illustration of the embodiment according to FIG. 1a, the first part of the shaft between the bell and the head plate being axially significantly shorter than the second part of the shaft between the stem and the head plate;

FIG. 1c is a three-dimensional illustration of the embodiment according to FIG. 1a, the first part of the shaft between the bell and the head plate being axially significantly longer than the second part of the shaft between the stem and the head plate;

FIG. 2a is a schematic side view of a further embodiment of the arrangement designed according to the invention, with a second fastening element designed as a slotted bell at one end of the shaft-shaped connecting element and a third fastening element designed as a clip at its other end, the two shaft parts between the bell and the head plate and between the head plate and the clip being of equal axial length;

FIG. 2b is a three-dimensional illustration of the embodiment according to FIG. 2a, the first part of the shaft between the bell and the head plate being axially significantly shorter than the second part of the shaft between the clip and the head plate;

FIG. 2c is a three-dimensional illustration of the embodiment according to FIG. 2a, the first part of the shaft between the bell and the head plate being axially significantly longer than the second part of the shaft between the clip and the head plate;

FIG. 3 is an embodiment of an arrangement designed according to the invention, in which both the second and the third fastening element are each designed as a slotted bell, the two parts of the shaft between the head plate and the two bells being of different lengths;

FIG. 4 is an embodiment of an arrangement designed according to the invention in which both the second and the third fastening element are designed as clips, the two parts of the shaft between the head plate and the two clips being of different lengths;

FIG. 5 is an embodiment of an arrangement designed according to the invention, in which both the second and the third fastening element are each designed as a stem, the two parts of the shaft between the head plate and the two stems being of different lengths;

FIG. 6 is an embodiment of an arrangement designed according to the invention, in which both the second and the third fastening elements are each designed as a piston, the two parts of the shaft between the head plate and the two pistons being of different lengths and the two pistons having different radial diameters;

FIG. 7a is an embodiment of the arrangement designed according to the invention, similar to FIG. 2a, but with a material recess encircling the shaft in an azimuthal manner above and below the head plate as predetermined breaking points;

FIG. 7b is an embodiment similar to FIG. 7a, but with a notch- or wedge-shaped, one-sided radial material recess in the shaft above and below the head plate as predetermined breaking points;

FIG. 8 is a three-dimensional detailed view of the first fastening element designed as a head plate, with a continuous shaft-shaped connecting element above and below the head plate;

FIG. 9 is a schematic side view of an embodiment of the arrangement according to the invention after the separation of the non-selected arrangement part and the formation a spike-shaped tip on the shaft in the region of the side of the head plate opposite the selected part (in this case, with a stem-shaped fastening element); and

FIG. 10 is a schematic side view of a separation tool formed as cutting pliers for severing the shaft-shaped connecting element and for forming a geometrically defined tip at the separation point between the selected part of the arrangement and the non-selected arrangement part.

### DETAILED DESCRIPTION OF THE PREFERRED EMBODIMENTS

The embodiments of the ossicular prosthesis 10; 20; 30; 40; 50; 60 (or parts thereof) according to the invention, shown schematically in the figures of the drawing, each comprise a first fastening element 11; 21; 31; 41; 51; 61 at one end, which is designed in the form of a flat head plate for mechanical contact with the tympanic membrane. On each end of the ossicular prosthesis 10; 20; 30; 40; 50; 60 sits a second fastening element 12; 22; 32; 42; 52; 62 for mechanically connecting the prosthesis to a member or parts of a member of the ossicular chain or to the inner ear. In between, along a longitudinal axis z, there is a shaft-shaped connecting element 14; 24; 34; 44; 54; 64 which connects the two fastening elements 11,12; 21,22; 31,32; 41,42 in a sound-conducting manner, the plate-shaped first fastening element 11; 21; 31; 41; 51; 61 having in its center a through-bore 15; 25 for receiving the shaft-shaped connecting element 14; 24; 34; 44; 54; 64, which is designed to be narrowable for fixing the connecting element 14; 24; 34; 44; 54; 64.

The invention is distinguished from known ossicular prostheses of the same type by the fact that that the shaft-shaped connecting element 14; 24; 34; 44; 54; 64 protrudes through the through-bore 15; 25; 35; 45 onto the side of the first fastening element 11; 21; 31; 41; 51; 61 opposite the second fastening element 12; 22; 32; 42; 52; 62, in that the connecting element 14; 24; 34; 44; 54; 64 has a third fastening element 13; 23; 33; 43; 53; 63 on its free end opposite the second fastening element 12; 22; 32; 42; 52; 62, designed for mechanical connection to a member or parts of a member of the ossicular chain or to the inner ear, and in that the shaft-shaped connecting element 14; 24; 34; 44; 54; 64 is designed, at least in its two regions adjacent to the through-bore 15; 25; 35; 45 of the first fastening element 11; 21; 31; 41; 51; 61 on both sides thereof in such a manner that it can be broken off above or below the plate-shaped first fastening element 11; 21; 31; 41; 51; 61 or can be separated by a separation tool 16.

This arrangement according to the invention gives the treating surgeon the opportunity to decide how the ossicular prosthesis should be designed immediately before the actual implantation. The non-selected part of the arrangement is then simply removed. The axial length of the ossicular prosthesis selected by the surgeon can be adjusted, for example, by sliding the first fastening element 11; 21; 31; 41; 51; 61, designed as a tympanic head plate, along the shaft-shaped connecting element 14; 24; 34; 44; 54; 64 and subsequently fastening or rigidifying it to the shaft, for example by means of a crimping process.

The arrangement according to the invention considerably reduces the necessary stockpiling of different auditory ossicular prostheses. The present invention offers the otologist a wide variety of options in the field of tympanoplasty.

In tympanoplasty, there are various options for coupling to the stapes, depending on the type and extent of the surgical procedure and the condition of the middle ear.

The most common methods are listed and explained below:
1. Ossiculoplasty:

Partial Ossicle Reconstruction Prosthesis (“PORP”):

This involves creating a prosthetic connection between the tympanic membrane and the stapes head. The stapes is largely intact.

The use of a PORP in tympanoplasty offers several advantages:
(a) Preservation of natural sound transmission: PORP allows more effective sound transmission because it uses the remaining intact parts of the auditory ossicles (especially the stapes), thus largely preserving physiological sound conduction.
(b) Better adaptation and stability: PORP can be easily adapted to the anatomical conditions of the middle ear, resulting in a more stable and secure connection. This reduces the risk of prosthesis dislocation or displacement compared to other reconstruction methods.
(c) Protection of the stapes footplate: PORP often results in less stress or manipulation of the stapes footplate, minimizing the risk of complications such as footplate dislocation or fractures.
(d) Improved hearing outcomes: Studies have shown that patients undergoing PORP tend to have better postoperative hearing improvements, especially in the mid and high frequency range, compared to other methods such as TORP.
(e) Versatility of application: PORP can be used in a variety of clinical situations, e.g., chronic otitis media where the stapes is intact.
(f) Lower risk of infection: The use of biocompatible materials such as titanium, which are often used in PORP prostheses, reduces the risk of postoperative infections and rejection reactions.

Overall, PORP offers an effective and reliable method for restoring sound conduction in the middle ear, while preserving the natural structures and functions of the ear as much as possible.
2. Total Ossicle Reconstruction Prosthesis (“TORP”): If the incus and malleus are completely missing, a prosthesis is used that creates a direct connection between the tympanic membrane and the footplate of the stapes.

The use of total ossicular reconstruction (TORP) in tympanoplasty offers several specific advantages:
(a) Restoration of sound transmission in severely damaged ossicular chains: TORP is particularly useful in cases where the incus and malleus had to be largely destroyed or removed. This method allows the restoration of sound transmission even in severely impaired middle ear structures.
(b) Direct connection between tympanic membrane and stapes footplate: TORP creates a direct connection between the tympanic membrane and the stapes footplate, allowing for efficient sound transmission. This can be particularly advantageous if the remaining parts of the auditory ossicles can no longer be used.
(c) Adaptation to individual anatomical conditions: TORP prostheses can be adjusted in length and shape to suit the patient's specific anatomical characteristics. This improves the fit and stability of the prosthesis.
(d) Reduced risk of ossicular prosthesis displacement: The direct and stable connection between the tympanic membrane and the stapes footplate reduces the risk of the prosthesis shifting or loosening, resulting in better hearing function in the long term.
(e) Bypassing damaged or non-functioning ossicles: In cases of significant damage or malfunction of the natural ossicular chain, TORP can bypass the damaged structures and still ensure effective sound transmission.

A particularly advantageous variant of TORP provides an oval stem for a larger contact surface. The applicant's “Regensburg” type total prosthesis made of titanium has a large contact region with the stirrup foot plate due to its oval and enlarged stem end. Together with a lower center of gravity, it ensures maximum balance and thus facilitates intraoperative handling.

Further advantageous features of this TORP: Small recesses in the head plate indicate the position of the stem. Below the head plate, the shaft has a diameter of only 0.2 mm. This makes it easy to bend, and it can be easily shaped into the desired final form.

In summary, TORP offers an effective solution for restoring sound conduction in the middle ear, especially in cases where the natural ossicular chain is severely compromised. The ability to create a stable and direct connection between the tympanic membrane and the stapes footplate often leads to good audiological outcomes and improved quality of life for patients.
3. Stapes footplate coupling:

Stapes prosthesis: This method is used in stapedotomy or stapedectomy, often in the treatment of otosclerosis. A prosthesis is anchored to the stapes footplate, either by insertion into a small opening (stapedotomy) or after removal of the stapes footplate (stapedectomy).

The use of a stapes prosthesis offers several advantages, particularly in the treatment of otosclerosis and other conditions that impede stapes movement. Here are some of the key benefits:
(a) Improved hearing performance: A stapes prosthesis can significantly improve sound transmission by bypassing the stiffness or fixation of the stapes and thus restoring the mechanical movement of sound waves from the middle ear to the cochlea.
(b) Minimal invasiveness: Modern techniques, such as stapedotomy, in which only a small hole is drilled into the footplate of the stapes, are minimally invasive and result in shorter recovery times and less postoperative discomfort compared to more extensive procedures.
(c) High success rate: The success rates for stapes prostheses are high, with a significant number of patients reporting a significant improvement in their hearing. Long-term results often show stable hearing improvement.
(d) Adaptability: Stapes prostheses are available in different sizes and materials, such as titanium or Teflon, which allows adaptation to the specific anatomical needs of the patient and ensures an optimal fit.
(e) Lower risk of complications: By using modern materials and techniques, the risk of complications such as infections or rejection reactions is low. In addition, the materials used are biocompatible and durable.
(f) Restoration of natural sound transmission: The stapes prosthesis allows almost natural sound transmission, resulting in better sound quality and speech intelligibility. This is particularly important for the quality of life of patients.
(g) Treatment of otosclerosis: In otosclerosis, a disease that causes ossification of the stapes and thus restricts its movement, the stapes prosthesis is a proven and effective treatment method to restore sound transmission.
(h) Long-term solution: A well-implanted stapes prosthesis can provide a permanent solution and reduce the need for further surgical intervention. Many patients report sustained improvements in their hearing over many years.
(i) Improved quality of life: Restoring hearing often significantly improves patients' quality of life. They can participate more in conversations and avoid social isolation, which will lead to an overall better sense of well-being.

In summary, the stapes prosthesis offers an effective and permanent solution for restoring hearing in patients with stapes fixation or otosclerosis, with a high success rate and low risks.

In order to offer the treating surgeon the most effective selection of decision options possible, the arrangement according to the invention can be designed in variants with different combinations of features:

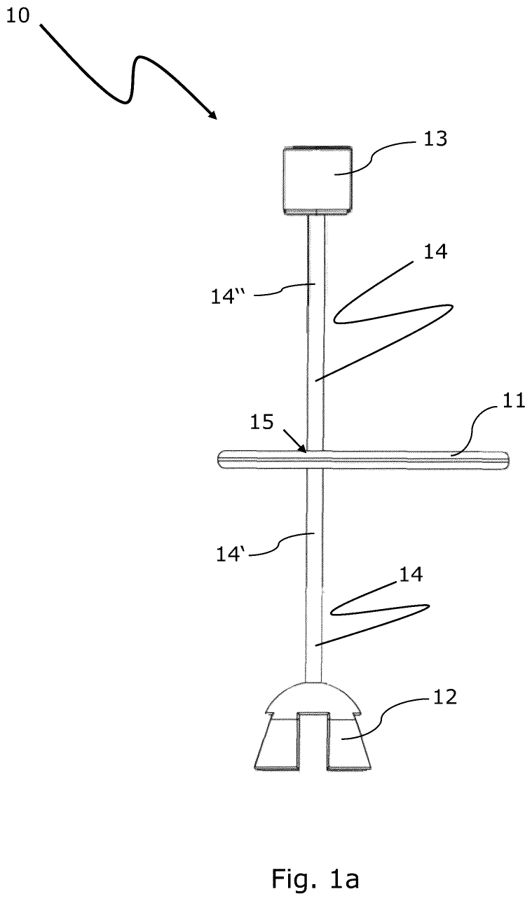

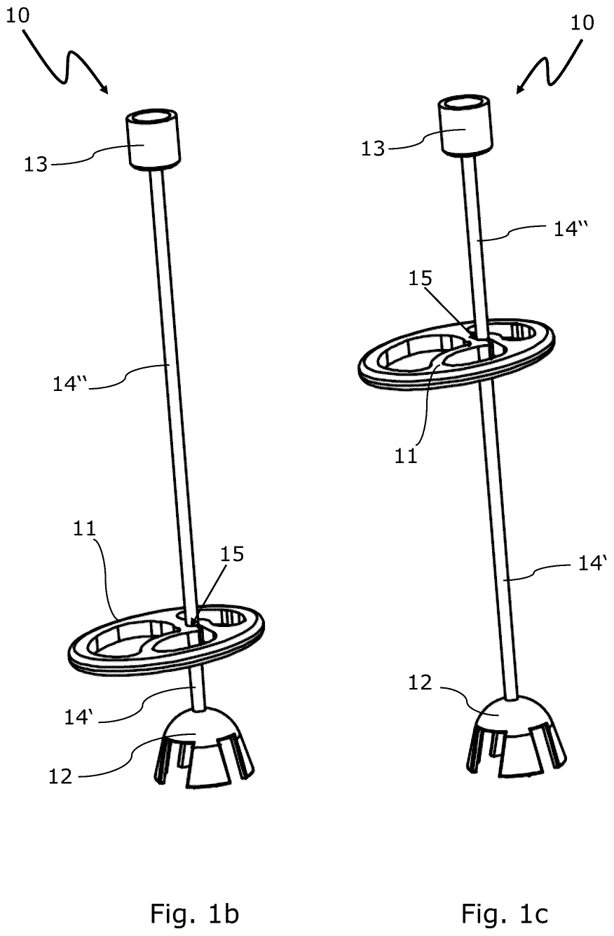

As such, as can be seen from FIGS. 1a to 1c, the first fastening element 11 together with the second fastening element 12 and with an intermediate first portion 14′ of the shaft-shaped connecting element 14 can form a partial prosthesis, while the first fastening element 11 together with the third fastening element 13 and with an intermediate second portion 14″ of the shaft-shaped connecting element 14 forms a total prosthesis.

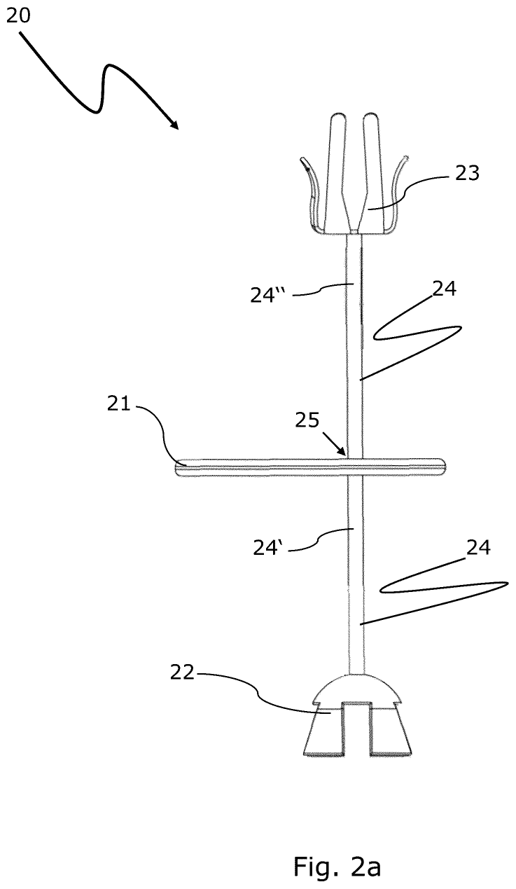

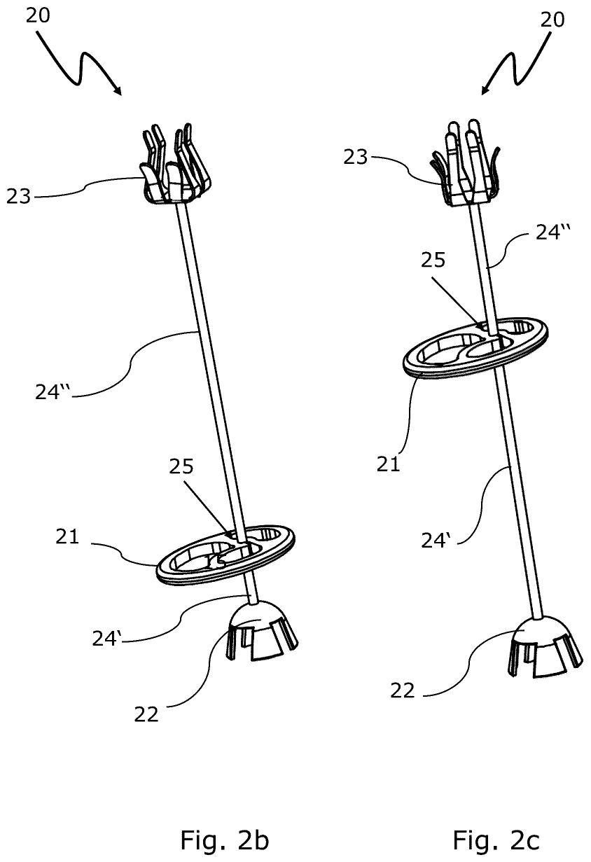

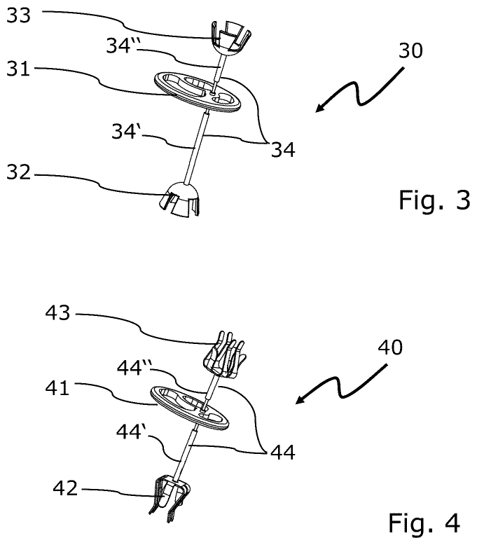

Alternatively, as shown in FIGS. 2a to 4, the first fastening element 21; 31; 41 together with the second fastening element 22; 32; 42 and with an intermediate first part 24′; 34′; 44′ of the shaft-shaped connecting element 24; 34; 44 can form a partial prosthesis, while the first fastening element 21; 31; 41 together with the third fastening element 23; 33; 43 and with an intermediate second part 24″; 34″; 44″ of the shaft-shaped connecting element 24; 34; 44 also forms a partial prosthesis.

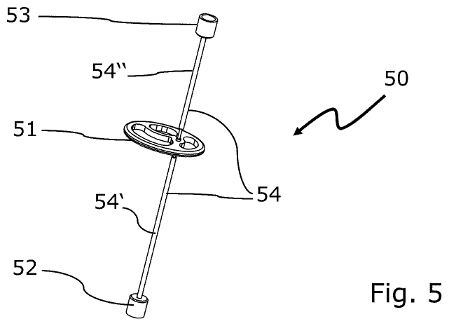

Another variant is shown in FIG. 5: It consists in connecting the first fastening element 51 together with the second fastening element 52 and with an intermediate first part 54′ of the shaft-shaped connecting element 54 to form a total prosthesis, the first fastening element 51 together with the third fastening element 53 and with an intermediate second part 54″ the shaft-shaped connecting element 54 also forming a total prosthesis.

In the last two variants of the invention, as in FIGS. 1b, 1c, 2b, 2c and 3 to 6, it can be advantageous if the first part 14′; 24′; 34′; 44′; 54′; 64′ of the shaft-shaped connecting element 14; 24; 34; 44; 54; 64 between the first fastening element 11; 21; 31; 41; 51; 61 and the second fastening element 12; 22; 32; 42; 52; 62 has a different axial length than the second part 14″; 24″; 34″; 44″; 54″; 64″ of the shaft-shaped connecting element 14; 24; 34; 44; 54; 64 between the first fastening element 11; 21; 31; 41; 51; 61 and the third fastening element 13; 23; 33; 43; 53; 63.

The specific design of the respective second fastening elements 12; 22; 32; 42; 52; 62 and the third fastening elements 13; 23; 33; 43; 53; 63 in the arrangement according to the invention also opens up a wide variety of possible variations:

The second fastening element 12; 22; 32 and/or the third fastening element 33 can be designed as a slotted bell, as shown in FIGS. 1a to 3 and 7a and 7b.

FIGS. 2a-c and 4 each show variants in which the second fastening element 42 and/or the third fastening element 23; 43 can be designed as a clip.

The second fastening element 52 and/or the third fastening element 13; 53 can also be stem-shaped, as FIGS. 1a-c, 5 and 9 show.

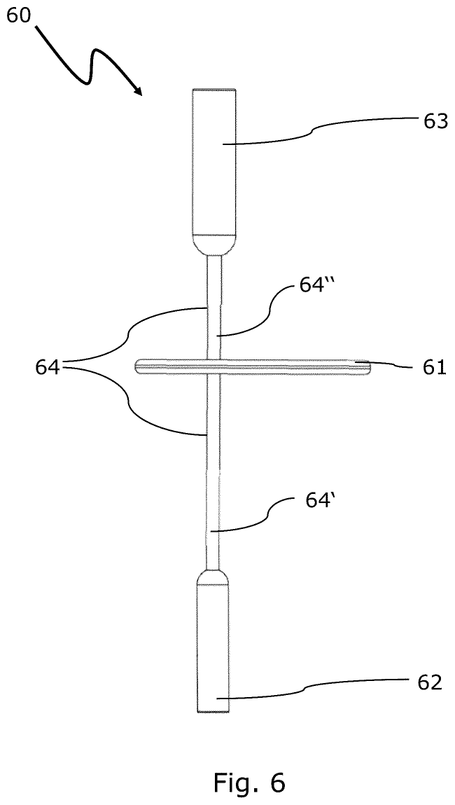

In the embodiment of FIG. 6, the second fastening element 62 and also the third fastening element 63 are even designed as a piston for connection to the inner ear.

As already explained above, the shaft-shaped connecting element 14; 24; 34; 44; 54; 64 must be designed, at least in its two regions adjacent to the through-bore 15; 25; 35; 45 of the first fastening element 11; 21; 31; 41; 51; 61 on both sides thereof, in such a way that it can be easily broken off above or below the plate-shaped first fastening element 11; 21; 31; 41; 51; 61 or separated by means of a separation tool 16.

A very simple design option to ensure this function can be to keep the radial thickness of the shaft-shaped connecting element 14; 24; 34; 44; 54; 64 sufficiently small. In this variant, the final length adjustment of the ossicular prosthesis selected by the surgeon can be achieved by simply moving the first fastening element 11; 21; 31; 41; 51; 61 axially along the shaft-shaped connecting element 14; 24; 34; 44; 54; 64 and then finally crimping the head plate at the desired shaft position.

A further break-off aid can be that the shaft-shaped connecting element 14; 24; 34; 44; 54; 64 has a predetermined breaking point 17′; 17″ in its two regions adjacent to the through-bore 15; 25; 35; 45 of the first fastening element 11; 21; 31; 41; 51; 61 on both sides thereof.

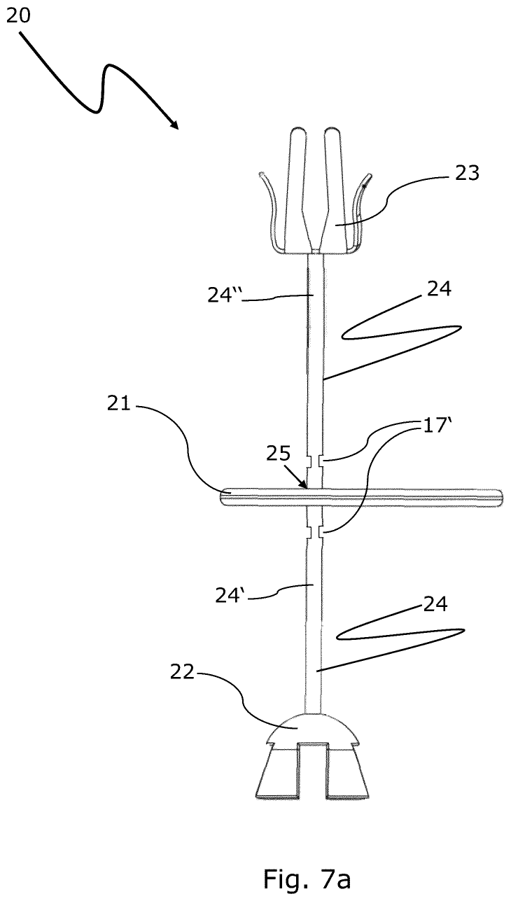

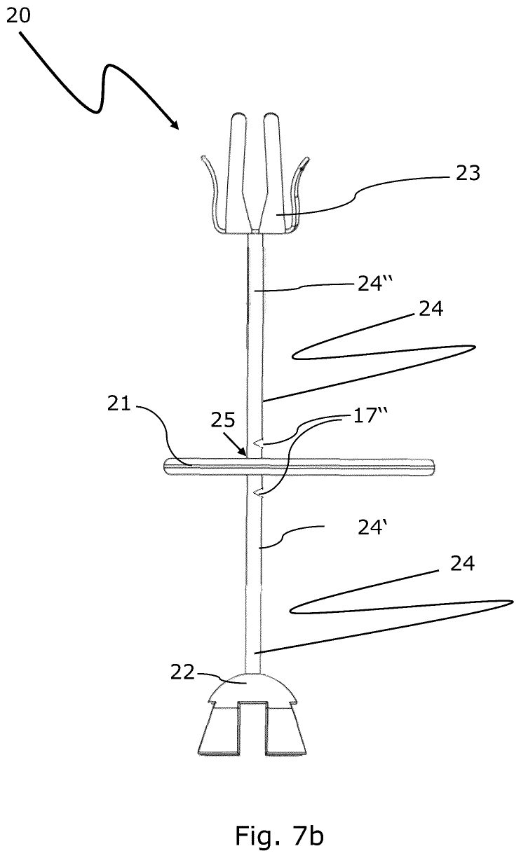

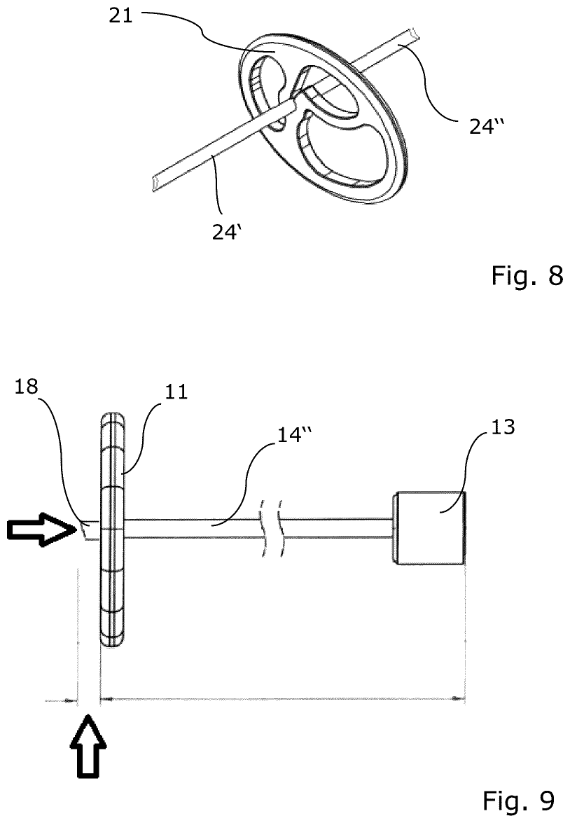

Such a predetermined breaking point 17′ can comprise an annular azimuthally circumferential material recess around the shaft-shaped connecting element 14; 24; 34; 44; 54; 64, as shown schematically in FIG. 7a. The predetermined breaking point 17″ can also be designed as a notch- or wedge-shaped, one-sided radial material recess in the shaft-shaped connecting element 14; 24; 34; 44; 54; 64, as illustrated in FIG. 7b.

After the removal of the non-selected part of the arrangement according to the invention by the treating surgeon immediately before the actual implantation of the desired ossicular prosthesis, there is initially a broken or cut end in the selected part at the shaft end of the connecting element above the tympanic head plate, which can be worked (e.g., removed or smoothed) before the prosthesis is inserted into the patient's middle ear. However, instead, this end can also be deliberately left in its current form, especially if a special tool was used to remove the non-selected part:

### Spike and Cartilage Disk

After adjusting the length of the prosthesis before implantation, it usually has a maximum length of 0.1 mm. When removing the non-selected part of the arrangement according to the invention (if necessary, with a special tool; see below), the broken or cut end can be deliberately shaped into a pointed “spike.” This spike, which protrudes beyond the tympanic head plate in the direction of the tympanic membrane, can then be used to fix a cartilage disk to the head plate (prosthesis head).

When using prostheses such as the PORP or TORP described above, a cartilage disk which has been previously surgically removed from the patient's body can be inserted between the head plate of the prosthesis and the tympanic membrane. This cartilage disk has several important functions and benefits:
(a) Stability and positioning: The cartilage disk helps to keep the prosthesis stable and secure its position. This reduces the risk of displacement or loosening of the prosthesis, resulting in better and more consistent sound transmission. (b) Prevention of tympanic membrane perforations: By inserting the cartilage disk, direct contact between the hard head plate of the prosthesis and the sensitive tympanic membrane is avoided. This reduces the risk of tympanic membrane perforation or damage that could occur due to mechanical pressure or movement of the prosthesis.
(c) Damping and vibration optimization: The cartilage disk acts as a damping element that optimizes the vibrations between the prosthesis and the tympanic membrane. This contributes to improved sound quality and helps avoid unpleasant noises or distortion.
(d) Biocompatibility and tissue compatibility: Cartilage is a biocompatible material that can be easily integrated into the tissue of the middle ear. This promotes healing and reduces the risk of inflammation or rejection compared to artificial materials.
(e) Avoidance of scarring and adhesions: The cartilage disk can help prevent the formation of scar tissue and adhesions that could impair the mobility of the prosthesis and sound transmission.
(f) Flexibility and adaptation to individual anatomy: Cartilage can be individually adjusted in size and shape to ensure optimal fit and function in the middle ear. This is particularly important in complex anatomical situations or in cases of previous surgery.
(g) Long-term durability: Cartilage is a robust material that remains stable over time and does not degenerate or break down as easily as other types of tissue. This contributes to the long-term functionality of the reconstructed ossicular chain.

In summary, the cartilage disk in both PORP and TORP serves as a multifunctional element that ensures the stability of the prosthesis, protects the tympanic membrane, optimizes vibration transmission, and promotes biocompatibility. The spike then ensures optimal mechanical coupling of the ossicular prosthesis to the cartilage disk. All these factors contribute to better surgical and audiological outcomes.

### Special Tool

The use of specially developed cutting forceps can also be extremely helpful for reworking the ossicular prosthesis that will ultimately be implanted in the middle ear. With their help, the surgeon removes the protruding prosthetic shaft on the lateral side of the head plate. A pin or spike is created which, when the prosthesis is inserted, fixes the transplant material, such as cartilage or fascia, inserted between the implant and the tympanic membrane.

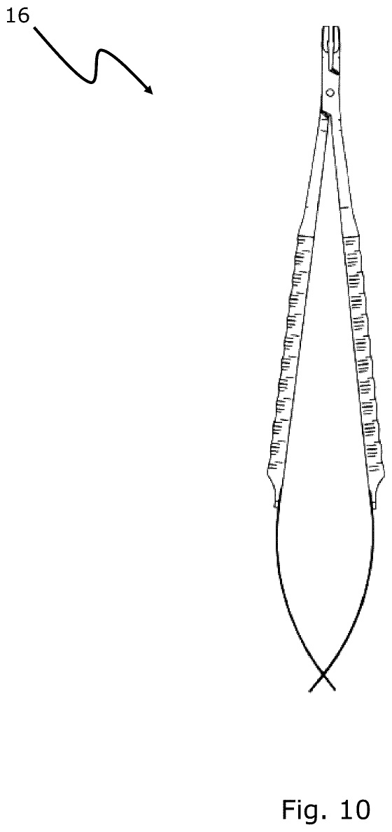

The present invention therefore also describes a system comprising an ossicular prosthesis 10; 20; 30; 40; 50; 60 of the type described above and a, as illustrated in FIG. 10, separation tool 16 for cutting through the shaft-shaped connecting element 14; 24; 34; 44; 54; 64. This separation tool 16 can be designed as cutting pliers for forming a geometrically defined tip at the separation point of the connecting element 14; 24; 34; 44; 54; 64, or also in the form of tweezers.

### LIST OF REFERENCE SIGNS

10; 20; 30; 40; 50; 60 ossicular prosthesis
11; 21; 31; 41; 51; 61 first fastening element
12; 22; 32; 42; 52; 62 second fastening element
13; 23; 33; 43; 53; 63 third fastening element
14; 24; 34; 44; 54; 64 connecting element
14′; 24′; 34′; 44′; 54′; 64′ first portion of the connecting element
14″; 24″; 34″; 44″; 54″; 64″ second portion of the connecting element
15; 25 through-bore
16 separation tool
17′; 17″ predetermined breaking point
z longitudinal axis

### LIST OF REFERENCES

Publications considered for the assessment of patentability:
[1] DE 298 19 892 U1≈EP 0 998 884 B1≈U.S. Pat. No. 6,387,128 B1
[2] DE 42 10 235 C1; EP 0 809 982 B1
[3] EP 1 181 907 B1
[4] US 2004/0162614 A1
[5] EP 1 833 424 B1
[6] EP 1 972 307 B1
[7] EP 3 311 773 B1
[8] DE 10 2007 041 539 B4
[9] EP 2 238 946 B1
[10] EP 2 601 909 B1
[11] EP 3 130 315 B1
[12] EP 1 961 400 B1
[13] DE 10 2022 119 451 B3
[14] DE 10 2009 006 047 B3
[15] DE 10 2020 108 887 A1
[16] US 2020/0113675 A1

What is claimed is:

1. An ossicular prosthesis which replaces or bridges at least one member or parts of a member of the ossicular chain, the ossicular prosthesis comprising:
a first fastening element designed as a flat head plate configured for mechanical engagement with the tympanic membrane;
a second fastening element configured for mechanical connection to a member or parts of a member of the auditory ossicular chain or to the inner ear; and
a shaft-shaped connecting element which connects the two fastening elements along a longitudinal axis in a manner conducting sound, the first fastening element having a through-bore in its center for receiving the shaft-shaped connecting element, which through-bore is designed to be narrowable configured for fixing the connecting element;
wherein the shaft-shaped connecting element protrudes through the through-bore onto the side of the first fastening element opposite the second fastening element;
wherein the connecting element carries, at its free end opposite the second fastening element, a third fastening element which is designed for mechanical connection to a member or parts of a member of the ossicular chain or to the inner ear; and
wherein the shaft-shaped connecting element is designed, at least in its two regions adjacent to the through-bore of the first fastening element on both sides thereof, wherein it is configured to be broken off above or below the first fastening element or separated by means of a separation tool.

2. The ossicular prosthesis according to claim 1, wherein the shaft-shaped connecting element has a predetermined breaking point in each of its two regions adjacent to the through-bore of the first fastening element on both sides thereof.

3. The ossicular prosthesis according to claim 2, wherein the predetermined breaking point comprises a material recess which runs azimuthally around the shaft-shaped connecting element in the shape of a ring.

4. The ossicular prosthesis according to claim 2, wherein the predetermined breaking point comprises a notch-shaped or wedge-shaped, one-sided radial material recess in the shaft-shaped connecting element.

5. The ossicular prosthesis according to claim 1, wherein the first fastening element together with the second fastening element and with a first part of the shaft-shaped connecting element located therebetween forms a partial prosthesis, and in that the first fastening element together with the third fastening element and with a second part of the shaft-shaped connecting element located therebetween forms a total prosthesis.

6. The ossicular prosthesis according to claim 1, wherein the first fastening element together with the second fastening element and with a first part of the shaft-shaped connecting element located therebetween forms a partial prosthesis, and in that the first fastening element together with the third fastening element and with a second part of the shaft-shaped connecting element located therebetween also forms a partial prosthesis.

7. The ossicular prosthesis according to claim 1, wherein the first fastening element together with the second fastening element and with a first part of the shaft-shaped connecting element located therebetween forms a total prosthesis, and in that the first fastening element together with the third fastening element and with a second part of the shaft-shaped connecting element located therebetween also forms a total prosthesis.

8. The ossicular prosthesis according to claim 6, wherein the first part of the shaft-shaped connecting element between the first fastening element and the second fastening element has a different axial length than the second part of the shaft-shaped connecting element between the first fastening element and the third fastening element.

9. The ossicular prosthesis according to claim 8, wherein the first part of the shaft-shaped connecting element has an axial length between 1.75 mm and 3.5 mm, and in that the second part of the shaft-shaped connecting element has an axial length between 3 mm and 7 mm.

10. The ossicular prosthesis according to claim 7, wherein the first part of the shaft-shaped connecting element between the first fastening element and the second fastening element has a different axial length than the second part of the shaft-shaped connecting element between the first fastening element and the third fastening element.

11. The ossicular prosthesis according to claim 10, wherein the first part of the shaft-shaped connecting element has an axial length between 1.75 mm and 3.5 mm, and in that the second part of the shaft-shaped connecting element has an axial length between 3 mm and 7 mm.

12. The ossicular prosthesis according to claim 1, wherein the shaft-shaped connecting element has a radial thickness transverse to the longitudinal axis between 0.1 mm and 0.2 mm.

13. The ossicular prosthesis according to claim 1, wherein the second fastening element and/or the third fastening element is designed as a slotted bell.

14. The ossicular prosthesis according to claim 1, wherein the second fastening element and/or the third fastening element is designed as a clip.

15. The ossicular prosthesis according to claim 1, wherein the second fastening element and/or the third fastening element is stem shaped.

16. The ossicular prosthesis according claim 1, wherein the second fastening element and/or the third fastening element is designed as a piston.

17. A system comprising the ossicular prosthesis according to claim 1, and the separation tool for severing the shaft-shaped connecting element, wherein the separation tool is designed as cutting pliers for forming a geometrically defined tip at the separation point of the connecting element, in the manner of tweezers.
